(19) United States
(12) Patent Application Publication
(10) Pub. No.: US 2025/0352366 A1
(43) Pub. Date: Nov. 20, 2025

(54) ENDOPROSTHESIS WITH IMPROVED CHARACTERISTICS

(71) Applicant: BOSTON SCIENTIFIC SCIMED, INC., Maple Grove, MN (US)

(72) Inventors: CELINE GLYNN, Galway (IE); GARY GILMARTIN, Foxford (IE); DANIEL TUCK, Galway (IE); FIONN STAPLETON, Galway (IE); THOMAS MARTIN KEATING, Galway (IE); HARRY MULLEADY, Galway (IE); JOHN THOMAS O'DRISCOLL, Galway (IE)

(73) Assignee: BOSTON SCIENTIFIC SCIMED, INC., Maple Grove, MN (US)

(21) Appl. No.: 19/208,869

(22) Filed: May 15, 2025

Related U.S. Application Data
(60) Provisional application No. 63/648,451, filed on May 16, 2024.

(51) Int. Cl. A61F 2/88

(52) U.S. Cl. CPC A61F 2/88; A61F 2210/0076; A61F 2230/0004; A61F 2250/001

(57) ABSTRACT

An endoprosthesis includes a body portion extending axially from a first end to a second end. The body portion is formed from a single wire extending from the first end to the second end. The single wire forms a plurality of circumferential rings. Each circumferential ring comprises an undulating arrangement of struts defining peaks and valleys. At least one circumferential ring may include a loop formed at each peak or at each valley of the at least one circumferential ring. A loop may be formed at each peak or at each valley of each circumferential ring. A loop may be formed at each peak and at each valley of each circumferential ring.

### CROSS-REFERENCE TO RELATED APPLICATIONS

The application claims the benefit of U.S. Provisional Patent Application Ser. No. 63/648,451, filed on May 16, 2024, the disclosure of which is incorporated herein by reference.

### TECHNICAL FIELD

The present disclosure pertains to medical devices, methods for manufacturing medical devices, and uses thereof. More particularly, the present disclosure pertains to an endoprosthesis or stent for implantation in a body lumen, and associated methods.

### BACKGROUND

An endoprosthesis may be used in the treatment of body lumens. One type of endoprosthesis used in the repair and/or treatment of diseases in various body lumens is a stent. A stent is a generally longitudinal tubular device formed of biocompatible material which is useful to open and support various lumens in the body. For example, stents may be used in the vascular system, urogenital tract, gastrointestinal tract, esophageal tract, tracheal/bronchial tubes, and bile duct, as well as in a variety of other applications in the body.

In some instances, it may be desirable to design an endoprosthesis to include sufficient flexibility and conformability to the body lumen, while maintaining sufficient radial force to open the body lumen at the treatment site and/or prevent migration of the endoprosthesis within the body lumen. In some instances, it may be desirable to reduce or limit foreshortening. In some instances, different endoprosthesis configurations may provide different deliverability, flexibility, conformability, radial force/strength, and/or anchoring/migration characteristics.

Of the known medical devices and methods, each has certain advantages and disadvantages. There is an ongoing need to provide alternative medical devices as well as alternative methods for manufacturing and using medical devices.

### SUMMARY

In one example, an endoprosthesis may comprise a body portion extending axially from a first end to a second end along a central longitudinal axis. The body portion may be formed from a single wire extending from the first end to the second end. The single wire may form a plurality of circumferential rings extending around the central longitudinal axis. Each circumferential ring of the plurality of circumferential rings may comprise an undulating arrangement of struts defining peaks and valleys. At least one circumferential ring of the plurality of circumferential rings may comprise a loop formed at each peak or at each valley of the at least one circumferential ring of the plurality of circumferential rings.

In addition, or alternatively, to any example disclosed herein, the plurality of circumferential rings extends helically around the central longitudinal axis.

In addition, or alternatively, to any example disclosed herein, the loop formed at each peak or at each valley is a closed loop formed by crossing the single wire over itself within the at least one circumferential ring of the plurality of circumferential rings.

In addition, or alternatively, to any example disclosed herein, endoprosthesis may comprise a polymeric covering coupled to the body portion.

In addition, or alternatively, to any example disclosed herein, a crossover point of the single wire forming the loop is disposed opposite an apex of its respective peak or valley.

In addition, or alternatively, to any example disclosed herein, the plurality of circumferential rings extends non-helically around the central longitudinal axis.

In addition, or alternatively, to any example disclosed herein, and in a second example, an endoprosthesis may comprise a body portion extending axially from a first end to a second end along a central longitudinal axis. The body portion may be formed from a single wire extending from the first end to the second end. The single wire may form a plurality of circumferential rings extending around the central longitudinal axis. Each circumferential ring of the plurality of circumferential rings may comprise an undulating arrangement of struts defining peaks and valleys. A loop may be formed at each peak or at each valley of each circumferential ring of the plurality of circumferential rings.

In addition, or alternatively, to any example disclosed herein, the plurality of circumferential rings extends helically around the central longitudinal axis.

In addition, or alternatively, to any example disclosed herein, the loop formed at each peak or at each valley is a closed loop formed by crossing the single wire over itself within each circumferential ring of the plurality of circumferential rings.

In addition, or alternatively, to any example disclosed herein, a crossover point of the single wire forming the closed loop is not disposed at an apex of its respective peak or valley.

In addition, or alternatively, to any example disclosed herein, a size of the closed loop is configured to increase when the endoprosthesis is radially constrained, and the size of the closed loop is configured to decrease when the endoprosthesis radially expands.

In addition, or alternatively, to any example disclosed herein, endoprosthesis may comprise a polymeric covering coupled to the body portion.

In addition, or alternatively, to any example disclosed herein, and in a third example, an endoprosthesis may comprise a body portion extending axially from a first end to a second end along a central longitudinal axis. The body portion may be formed from a single wire extending from the first end to the second end. The single wire may form a plurality of circumferential rings extending around the central longitudinal axis. Each circumferential ring of the plurality of circumferential rings may comprise an undulating arrangement of struts defining peaks and valleys. A loop may be formed at each peak and at each valley of each circumferential ring of the plurality of circumferential rings.

In addition, or alternatively, to any example disclosed herein, loops formed at each peak of each circumferential ring are axially aligned with loops formed at each peak of each immediately adjacent circumferential ring parallel to the central longitudinal axis, and loops formed at each valley of each circumferential ring are axially aligned with loops formed at each valley of each immediately adjacent circumferential ring parallel to the central longitudinal axis.

In addition, or alternatively, to any example disclosed herein, loops formed at each peak of each circumferential ring are helically aligned around the central longitudinal axis with loops formed at each peak of each immediately adjacent circumferential ring, and loops formed at each valley of each circumferential ring are helically aligned around the central longitudinal axis with loops formed at each valley of each immediately adjacent circumferential ring.

In addition, or alternatively, to any example disclosed herein, the loop formed at each peak and at each valley of each circumferential ring is formed by the single wire extending at least 360 degrees around a center of the loop.

In addition, or alternatively, to any example disclosed herein, the loop formed at each peak and at each valley of each circumferential ring is formed by the single wire extending at least 450 degrees around the center of the loop.

In addition, or alternatively, to any example disclosed herein, the single wire extends in a first circumferential direction around the center of the loop at each peak and in a second circumferential direction opposite the first circumferential direction around the center of the loop at each valley.

In addition, or alternatively, to any example disclosed herein, a crossover point of the single wire forming the loop is disposed at an apex of its respective peak or valley.

In addition, or alternatively, to any example disclosed herein, the endoprosthesis may comprise a polymeric covering coupled to the body portion.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIGS. 1-3 are side views illustrating selected aspects of an endoprosthesis in accordance with the disclosure;

FIGS. 4-6 are side views illustrating selected aspects of an endoprosthesis in accordance with the disclosure; and

FIGS. 7-9 are side views illustrating selected aspects of an endoprosthesis in accordance with the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

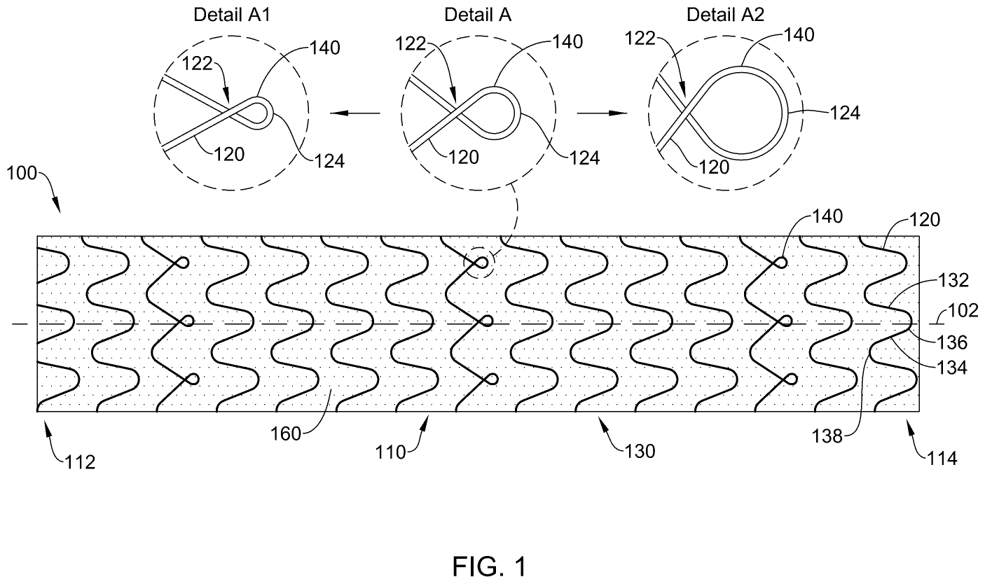

FIGS. 1-3 illustrate selected aspects of an example configuration of an endoprosthesis 100, such as a stent. The endoprosthesis 100 may comprise a body portion 110 extending axially from a first end 112 to a second end 114 along a central longitudinal axis 102. In some embodiments, the first end 112 and the second end 114 may define a length of the endoprosthesis 100 and/or the body portion 110. In at least some embodiments, the body portion 110 may be tubular. The body portion 110 may define a lumen extending therethrough from the first end 112 to the second end 114.

In some embodiments, the endoprosthesis 100 may comprise a flared first end portion (not shown) and/or a flared second end portion (not shown), wherein an outer diameter of the flared first end portion is greater than an outer diameter of the body portion 110 and/or an outer diameter of the flared second end portion is greater than the outer diameter of the body portion 110. Other configurations are also contemplated. The flared first end portion may be disposed proximate and/or adjacent the first end 112. In some embodiments, the flared first end portion may extend from the first end 112 to the body portion 110. The flared second end portion may be disposed proximate and/or adjacent the second end 114. In some embodiments, the flared second end portion may extend from the second end 114 to the body portion 110.

In some embodiments, the outer diameter of the body portion 110 may be generally uniform and/or generally constant along the length of the endoprosthesis 100 and/or the body portion 110. In some embodiments, the outer diameter of the body portion 110 may be generally uniform and/or generally constant along the length of the body portion 110 except for the flared first end portion and/or the flared second end portion, where present. In some embodiments, the outer diameter of the body portion 110 may be generally uniform and/or generally constant along the length of the body portion 110 from the flared first end portion to the flared second end portion, where present. In some embodiments, the outer diameter of the body portion 110 may be between about 4 millimeters and about 40 millimeters, between about 6 millimeters and about 35 millimeters, between about 8 millimeters and about 30 millimeters, between about 10 millimeters and about 25 millimeters, etc. Other configurations are also contemplated.

In some embodiments, the endoprosthesis 100 and/or the body portion 110 may be formed from a single wire 120 extending from the first end 112 to the second end 114. In some embodiments, the flared first end portion and/or the flared second end portion, where present, may be formed from the single wire 120. The single wire 120 may extend circumferentially around the central longitudinal axis 102 and/or the single wire 120 may encircle the central longitudinal axis 102. The single wire 120 is and/or consists of only one wire.

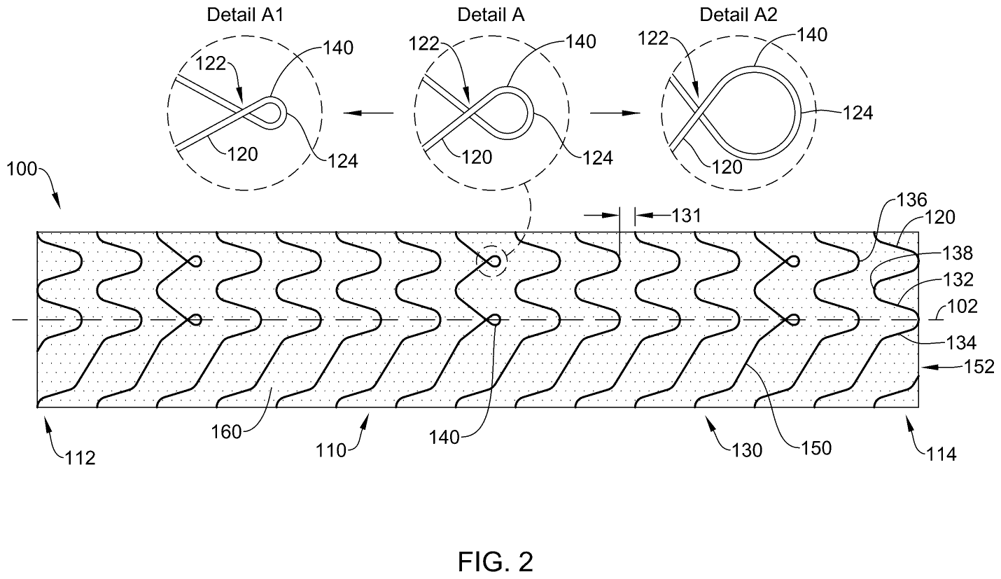

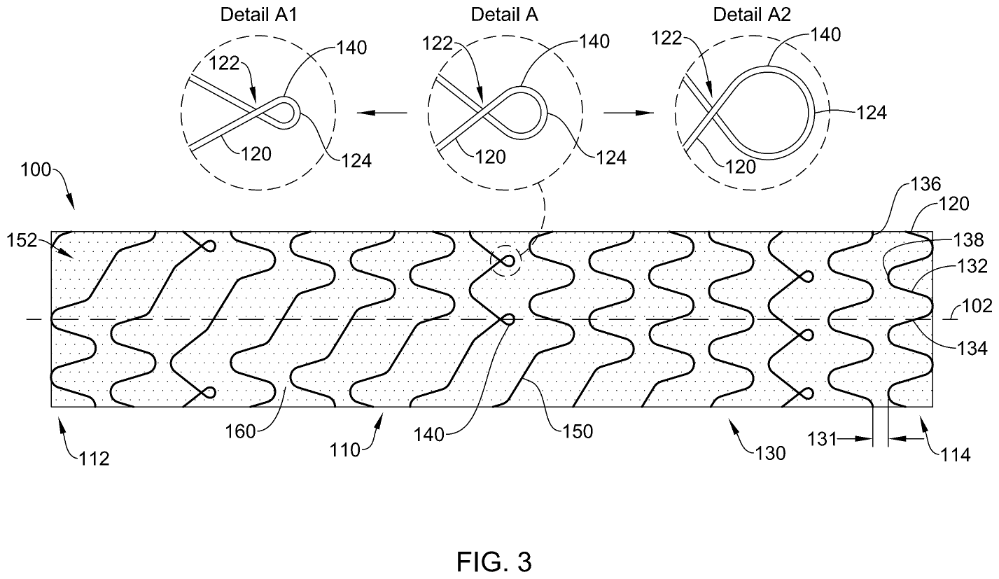

In some embodiments, the single wire 120 may form a plurality of circumferential rings 130 extending around the central longitudinal axis 102. In some embodiments, the plurality of circumferential rings 130 may extend helically around the central longitudinal axis 102, as seen in FIG. 1. In some embodiments, the plurality of circumferential rings 130 may extend non-helically around the central longitudinal axis 102, as seen in FIGS. 2-3. In some embodiments, the plurality of circumferential rings 130 may be substantially parallel to each other. Some suitable but non-limiting examples of materials for the endoprosthesis 100 and/or the single wire 120, such as metallic materials, composite materials, shape memory materials, combinations thereof, etc., are discussed below. In one non-limiting example, the endoprosthesis 100 and/or the single wire 120 may be formed from nickel-titanium alloy (e.g., nitinol). In another non-limiting example, the endoprosthesis 100 and/or the single wire 120 may be formed from nickel-titanium alloy with a platinum core. Other configurations and/or materials are also contemplated.

In some embodiments, the plurality of circumferential rings 130 may comprise a first end circumferential ring forming the first end 112, a second end circumferential ring forming the second end 114, and at least one medial circumferential ring disposed axially between the first end circumferential ring and the second end circumferential ring. In some embodiments, the at least one medial circumferential ring may comprise one circumferential ring, two circumferential rings, three circumferential rings, etc. up to a desired number of circumferential rings producing a desired overall length for the endoprosthesis 100.

In some embodiments, each circumferential ring of the plurality of circumferential rings 130 may comprise an undulating arrangement (e.g., a sinusoidal arrangement) of first struts 132 and second struts 134 defining peaks 136 and valleys 138. In at least some embodiments, within any particular circumferential ring of the plurality of circumferential rings 130, the first struts 132 have a first length defined by the peaks 136 and the valleys 138 and the second struts 134 have a second length defined by the peaks 136 and the valleys 138, wherein the first length is equal to the second length. In some embodiments, the first length and the second length may be uniform and/or constant among the plurality of circumferential rings 130. In some alternative embodiments, the first length and the second length may vary among the plurality of circumferential rings 130. In some embodiments, the first length and/or the second length may be between about 1 millimeter and about 15 millimeters. In some embodiments, the first length and/or the second length may be between about 3 millimeters and about 12 millimeters. In some embodiments, the first length and/or the second length may be between about 5 millimeters and about 10 millimeters. Other configurations are also contemplated.

In some embodiments, at least one circumferential ring of the plurality of circumferential rings 130 may comprise a loop 140 formed at each peak (ref. 136) or at each valley (ref. 138) of the at least one circumferential ring of the plurality of circumferential rings 130. For the purpose of illustration only, the figures show a loop 140 formed at each peak (ref. 136) of the at least one circumferential ring of the plurality of circumferential rings 130. However, it shall be understood that in some embodiments, a loop 140 may be formed at each valley (ref. 138) of the at least one circumferential ring of the plurality of circumferential rings 130. In addition, or alternatively, in some embodiments, at least one circumferential ring of the plurality of circumferential rings 130 may comprise a loop 140 formed at each peak (ref. 136) and at least one circumferential ring of the plurality of circumferential rings 130 may comprise a loop 140 formed at each valley (ref. 138). As such, embodiments are contemplated that comprise loops formed at peaks of one circumferential ring of the plurality of circumferential rings 130 and loops formed at valleys of a different circumferential ring of the plurality of circumferential rings 130. Other configurations are also contemplated.

In some alternative embodiments, at least one circumferential ring of the plurality of circumferential rings 130 may comprise a loop 140 formed at some peaks 136 (e.g., less than all peaks 136, or not at each/every peak) or at some valleys 138 (e.g., less than all valleys 138, or not at each/every valley) of the at least one circumferential ring of the plurality of circumferential rings 130. For example, the at least one circumferential ring of the plurality of circumferential rings 130 may comprise a loop 140 formed at every other or every second peak (ref. 136) or at every other or every second valley (ref. 138) of the at least one circumferential ring of the plurality of circumferential rings 130. Other configurations (e.g., every third peak/valley, etc.), including combinations thereof, are also contemplated.

In some embodiments, each circumferential ring of the at least one circumferential ring of the plurality of circumferential rings 130 may be spaced apart from another circumferential ring of the at least one circumferential ring of the plurality of circumferential rings 130. In some embodiments, the at least one circumferential ring of the plurality of circumferential rings 130 may comprise exactly one circumferential ring, two circumferential rings, three circumferential ring, four circumferential rings, six circumferential rings, eight circumferential rings, or another suitable number of circumferential rings.

In some embodiments, the loop 140 formed at each peak (ref. 136) or at each valley (ref. 138) of the at least one circumferential ring of the plurality of circumferential rings 130 is a closed loop that may be formed by crossing the single wire 120 over itself within the at least one circumferential ring of the plurality of circumferential rings 130, as seen in Detail A. In some embodiments, a crossover point 122 of the single wire 120 forming the loop 140 and/or the closed loop may not be disposed at an apex 124 of its respective peak (ref. 136) or valley (ref. 138). In some embodiments, the (each) crossover point 122 of the single wire 120 forming the (each) loop 140 and/or the closed loop may be disposed opposite the apex 124 of its respective peak (ref. 136) or valley (ref. 138).

In at least some embodiments, a size (e.g., a perimeter and/or an internal area) of the loop 140 and/or the closed loop may be configured to increase when the endoprosthesis 100 and/or the body portion 110 is radially constrained (e.g., a collapsed delivery configuration), as shown in Detail A2, and the size (e.g., the perimeter and/or the internal area) of the loop 140 and/or the closed loop may be configured to decrease when the endoprosthesis 100 and/or the body portion 110 radially expands (e.g., an expanded deployed configuration), as shown in Detail A1. In some embodiments, the single wire 120 may be configured to translate and/or slide over and/or along itself such that the (each) crossover point 122 moves axially (e.g., toward the first end 112 and/or the second end 114) as the endoprosthesis 100 and/or the body portion 110 shifts between the collapsed delivery configuration and the expanded deployed configuration.

In some embodiments, the peaks 136 and the valleys 138 of adjacent circumferential rings of the plurality of circumferential rings 130 may be aligned with each other axially along the length of the body portion 110 and/or the endoprosthesis 100, as seen in FIG. 2. For example, the peaks 136 of one circumferential ring of the plurality of circumferential rings 130 may be aligned axially with the peaks 136 of an immediately adjacent circumferential ring of the plurality of circumferential rings 130. Similarly, the valleys 138 of one circumferential ring of the plurality of circumferential rings 130 may be aligned axially with the valleys 138 of an immediately adjacent circumferential ring of the plurality of circumferential rings 130. In some embodiments, the peaks 136 and the valleys 138 of adjacent circumferential rings of the plurality of circumferential rings 130 may be aligned parallel to the central longitudinal axis 102. Other configurations are also contemplated.

In some embodiments, the peaks 136 and the valleys 138 of each circumferential ring of the plurality of circumferential rings 130 may be aligned with each other axially along the length of the body portion 110 and/or the endoprosthesis 100. For example, the peaks 136 of each circumferential ring of the plurality of circumferential rings 130 may be aligned axially with the peaks 136 of each other circumferential ring of the plurality of circumferential rings 130. Similarly, the valleys 138 of each circumferential ring of the plurality of circumferential rings 130 may be aligned axially with the valleys 138 of each other circumferential ring of the plurality of circumferential rings 130. In some embodiments, the peaks 136 and the valleys 138 of each circumferential ring of the plurality of circumferential rings 130 may be aligned parallel to the central longitudinal axis 102. Other configurations are also contemplated.

In some embodiments, each adjacent pair of circumferential rings of the plurality of circumferential rings 130 may define an axial spacing 131 therebetween, as seen in FIGS. 2-3. The axial spacing 131 may be measured axially and/or parallel to the central longitudinal axis 102. In some embodiments, the axial spacing 131 may be defined between the valleys 138 of one circumferential ring and the peaks 136 of an immediately adjacent circumferential ring. In some embodiments, the axial spacing 131 between adjacent pairs of circumferential rings of the plurality of circumferential rings 130 may be uniform along the length of the body portion 110 and/or the endoprosthesis 100. In some embodiments, adjacent pairs of circumferential rings of the plurality of circumferential rings 130 are equally and/or uniformly spaced apart along the length of the body portion 110 and/or the endoprosthesis 100. In some embodiments, the axial spacing 131 between adjacent pairs of circumferential rings of the plurality of circumferential rings 130 may vary along the length of the body portion 110 and/or the endoprosthesis 100. In some embodiments, adjacent pairs of circumferential rings of the plurality of circumferential rings 130 are unequally and/or non-uniformly spaced apart along the length of the body portion 110 and/or the endoprosthesis 100. In some embodiments, the axial spacing 131 between adjacent pairs of circumferential rings of the plurality of circumferential rings 130 may increase along the length of the body portion 110 and/or the endoprosthesis 100 from the first end 112 toward and/or to the second end 114. Alternatively, in some embodiments, the axial spacing 131 between adjacent pairs of circumferential rings of the plurality of circumferential rings 130 may decrease along the length of the body portion 110 and/or the endoprosthesis 100 from the first end 112 toward and/or to the second end 114.

In some embodiments, the peaks 136 and the valleys 138 of each circumferential ring of the plurality of circumferential rings 130 may define a circumferential ring height for that circumferential ring measured parallel to the central longitudinal axis 102. In some embodiments, the circumferential ring height may be uniform along the length of the body portion 110 and/or the endoprosthesis 100 and/or among the plurality of circumferential rings 130, as illustrated in the figures. While not expressly shown, in some embodiments, the circumferential ring height may vary along the length of the body portion 110 and/or the endoprosthesis 100 and/or among the plurality of circumferential rings 130. In some embodiments, the circumferential ring height may increase along the length of the body portion 110 and/or the endoprosthesis 100 and/or among the plurality of circumferential rings 130 from the first end 112 toward and/or to the second end 114. Alternatively, in some embodiments, the circumferential ring height may decrease along the length of the body portion 110 and/or the endoprosthesis 100 and/or among the plurality of circumferential rings 130 from the first end 112 toward and/or to the second end 114.

In some embodiments, each circumferential ring of the plurality of circumferential rings 130 may be connected to an axially adjacent circumferential ring of the plurality of circumferential rings 130 by a transition segment 150 of the single wire 120, as seen in FIGS. 2-3 for example. In at least some embodiments, each circumferential ring of the plurality of circumferential rings 130 may be discontinuous and/or may extend around less than a full circumference of the endoprosthesis 100 and/or the body portion 110. Instead, each circumferential ring of the plurality of circumferential rings 130 may have a first end and a second end, wherein the transition segment 150 extends between a first end of one circumferential ring of the plurality of circumferential rings 130 and a second end of an immediately adjacent circumferential ring of the plurality of circumferential rings 130 (or the transition segment 150 extends between a second end of one circumferential ring of the plurality of circumferential rings 130 and a first end of an immediately adjacent circumferential ring of the plurality of circumferential rings 130). In some embodiments, at least a portion of the first end circumferential ring and/or the second end circumferential ring may extend completely around the full circumference of the endoprosthesis 100 and/or the body portion 110, and the at least one medial circumferential ring may be discontinuous and/or may extend around less than the full circumference of the endoprosthesis 100 and/or the body portion 110.

In some embodiments, the first struts 132 may be disposed at a first angle relative to the central longitudinal axis 102 in a side view and/or a flat pattern view of the body portion 110 and/or the endoprosthesis 100, and the second struts 134 may be disposed at a second angle relative to the central longitudinal axis 102 in the side view and/or the flat pattern view of the body portion 110 and/or the endoprosthesis 100. In at least some embodiments, the second angle may be different from the first angle. In some embodiments, the first angle and/or the second angle may be between zero degrees and 90 degrees. In some embodiments, the first angle and/or the second angle may be between 5 degrees and 60 degrees. In some embodiments, the first angle and/or the second angle may be between 10 degrees and 45 degrees. In some embodiments, the first angle and/or the second angle may be between 15 degrees and 30 degrees. Other configurations are also contemplated.

The transition segment 150 of the single wire 120 may be disposed at a third angle relative to the central longitudinal axis 102 in the side view and/or the flat pattern view of the body portion 110 and/or the endoprosthesis 100. In at least some embodiments, the third angle may be different from the first angle and the second angle. In some embodiments, the transition segment 150 may have a length between about 5 millimeters and about 20 millimeters. In some embodiments, the transition segment 150 may have a length between about 8 millimeters and about 17 millimeters. In some embodiments, the transition segment 150 may have a length between about 10 millimeters and about 15 millimeters. Other configurations are also contemplated. In some embodiments, the third angle may be between about 30 degrees and about 90 degrees. In some embodiments, the third angle may be between about 35 degrees and about 75 degrees. In some embodiments, the third angle may be between about 40 degrees and about 60 degrees. Other configurations are also contemplated.

In some embodiments, each circumferential ring of the plurality of circumferential rings 130 may be connected to an axially adjacent circumferential ring of the plurality of circumferential rings 130 by a transition segment 150 of the single wire 120 disposed nonparallel to the central longitudinal axis 102, the first struts 132, and the second struts 134 in the side view and/or the flat pattern view of the body portion 110 and/or the endoprosthesis 100. Other configurations are also contemplated.

In some embodiments, the transition segment 150 extending and/or disposed between axially adjacent circumferential rings of the plurality of circumferential rings 130 may extend between a valley (ref. 138) of one circumferential ring and a peak (ref. 136) of an immediately axially adjacent circumferential ring (or between a peak of one circumferential ring and a valley of an immediately axially adjacent circumferential ring). As illustrated, the transition segment(s) 150 extend from a peak (ref. 136) of a first circumferential ring disposed closer to the first end 112 to a valley (ref. 138) of a second circumferential ring immediately axially adjacent the first circumferential ring that is disposed closer to the second end 114 than the first circumferential ring. Other configurations are also contemplated. In some embodiments, the transition segment 150 may be disposed nonparallel with the first struts 132 and the second struts 134 of one circumferential ring (e.g., the first circumferential ring) and the first struts 132 and the second struts 134 of the immediately axially adjacent circumferential ring (e.g., the second circumferential ring). In some embodiments, the transition segment 150 may be a substantially straight or linear segment extending between a valley (ref. 138) of one circumferential ring and a peak (ref. 136) of an immediately axially adjacent circumferential ring (or between a peak of one circumferential ring and a valley of an immediately axially adjacent circumferential ring). In some embodiments, the transition segment 150 may be a non-linear segment extending between a valley (ref. 138) of one circumferential ring and a peak (ref. 136) of an immediately axially adjacent circumferential ring (or between a peak of one circumferential ring and a valley of an immediately axially adjacent circumferential ring)—for example, the transition segment 150 may have a bend formed along its length between two short linear segments. Other configurations are also contemplated.

In some embodiments, all transition segments (e.g., an array of transition segments consisting of each transition segment 150 connecting and/or extending between adjacent circumferential rings of the plurality of circumferential rings 130) of the single wire 120 may collectively define a transition zone 152. In some embodiments, the transition zone 152 may extend and/or may be arranged parallel to the central longitudinal axis 102, as seen in FIG. 2. In some embodiments, each transition segment 150 of the single wire 120 may be aligned axially along the length of the body portion 110 and/or the endoprosthesis 100. In some embodiments, the transition zone 152 may extend and/or may be arranged helically along and/or around the central longitudinal axis 102, as seen in FIG. 3.

In some embodiments, the peaks 136 and the valleys 138 of adjacent circumferential rings of the plurality of circumferential rings 130 may extend and/or may be arranged helically along the length of the body portion 110 and/or the endoprosthesis 100. In some embodiments, the peaks 136 and the valleys 138 of adjacent circumferential rings of the plurality of circumferential rings 130 may extend and/or may be arranged helically along and/or around the central longitudinal axis 102. In some embodiments, the peaks 136 and the valleys 138 of each circumferential ring of the plurality of circumferential rings 130 may extend and/or may be arranged helically along the length of the body portion 110 and/or the endoprosthesis 100. In some embodiments, the peaks 136 and the valleys 138 of each circumferential ring of the plurality of circumferential rings 130 may extend and/or may be arranged helically along and/or around the central longitudinal axis 102. Other configurations are also contemplated.

In some embodiments, the endoprosthesis 100 may comprise a polymeric covering 160, shown in the figures via dotted shading, coupled to the body portion 110, the single wire 120, and/or the plurality of circumferential rings 130. In some embodiments, the polymeric covering 160 may be fixedly attached to the body portion 110, the single wire 120, and/or the plurality of circumferential rings 130.

In some embodiments, the polymeric covering 160 may extend along an inner surface of the endoprosthesis 100, the body portion 110, the single wire 120, and/or the plurality of circumferential rings 130. In some embodiments, the polymeric covering 160 may extend along an outer surface of the endoprosthesis 100, the body portion 110, the single wire 120, and/or the plurality of circumferential rings 130. In at least some embodiments, the body portion 110, the single wire 120, and/or the plurality of circumferential rings 130 may be embedded within the polymeric covering 160. Other configurations, including combinations thereof, are also contemplated. Some suitable but non-limiting examples of polymeric materials for the polymeric covering 160 are discussed below.

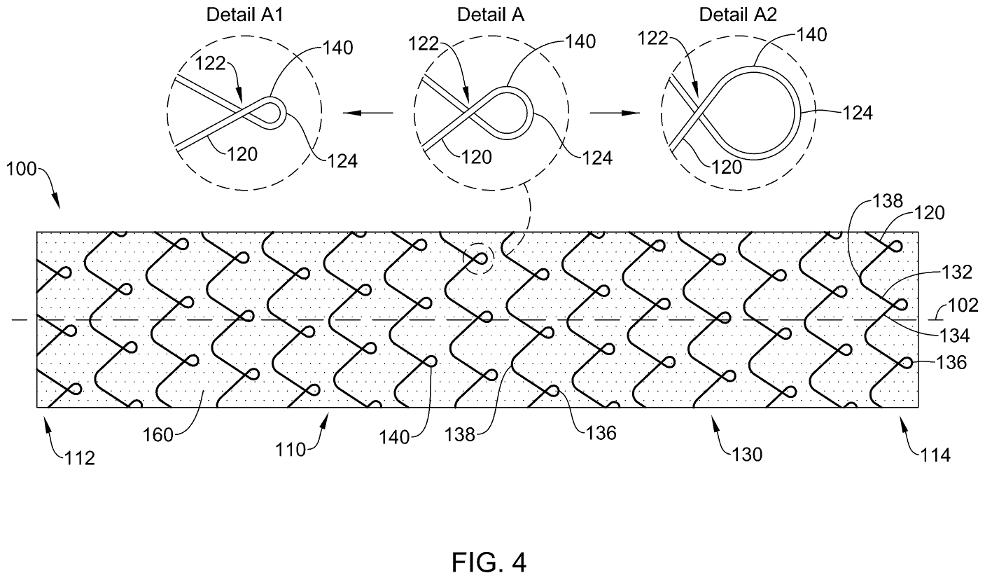

FIGS. 4-6 illustrate selected aspects of another example configuration of the endoprosthesis 100. At least some aspects that may be the same as or substantially similar to those described above and/or herein are not repeated in the interest of brevity. Reference numbers appearing in the figures that are not explicitly discussed with respect to that figure are shown for reference and the reader may refer to the description above and/or herein. Additionally, reference numbers discussed in the description that do not appear in the figures may be shown in other figures herein.

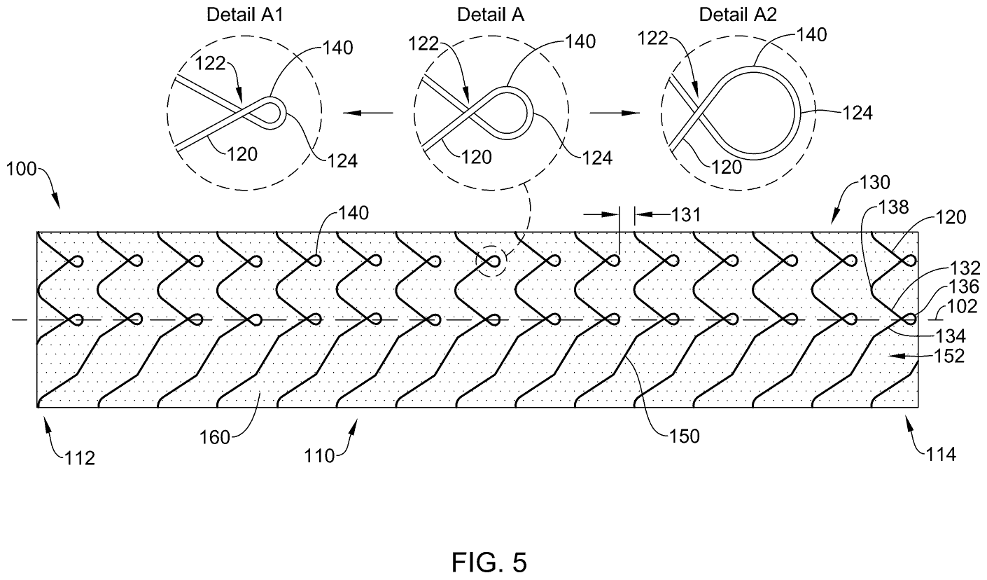

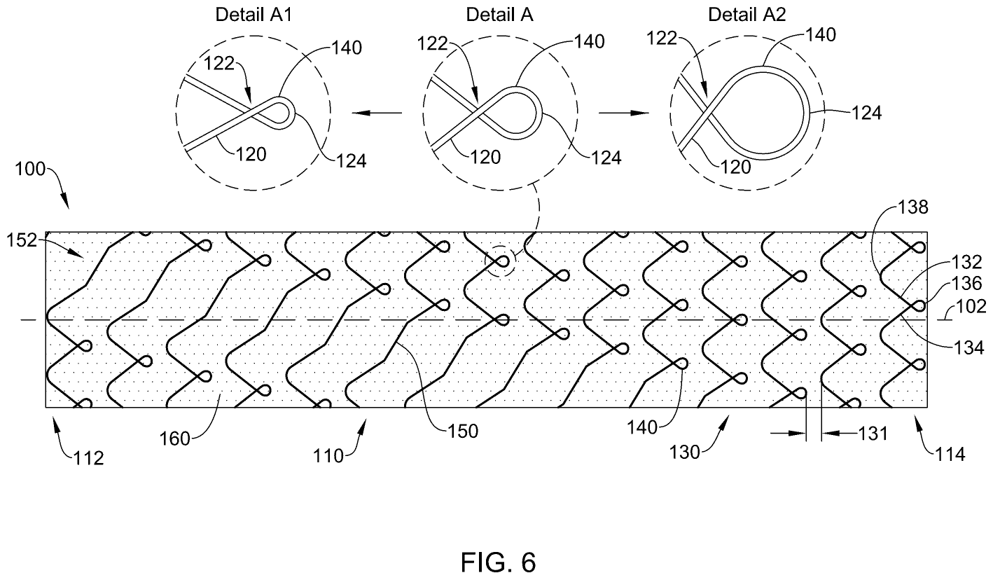

Similar to above, the endoprosthesis 100 and/or the body portion 110 may be formed from a single wire 120 extending from the first end 112 to the second end 114. In some embodiments, the single wire 120 may form a plurality of circumferential rings 130 extending around the central longitudinal axis 102. In some embodiments, the plurality of circumferential rings 130 may extend helically around the central longitudinal axis 102, as seen in FIG. 4. In some embodiments, the plurality of circumferential rings 130 may extend non-helically around the central longitudinal axis 102, as seen in FIGS. 5-6. In some embodiments, the plurality of circumferential rings 130 may be substantially parallel to each other.

In some embodiments, a loop 140 may be formed at each peak or at each valley of each circumferential ring of the plurality of circumferential rings 130. For the purpose of illustration only, the figures show a loop 140 formed at each peak (ref. 136) of each circumferential ring of the plurality of circumferential rings 130. However, it shall be understood that in some embodiments, a loop 140 may be formed at each valley (ref. 138) of each circumferential ring of the plurality of circumferential rings 130. In addition, or alternatively, in some embodiments, at least one circumferential ring of the plurality of circumferential rings 130 may comprise a loop 140 formed at each peak (ref. 136) and at least one circumferential ring of the plurality of circumferential rings 130 may comprise a loop 140 formed at each valley (ref. 138). As such, embodiments are contemplated that comprise loops formed at peaks of one or more circumferential rings of the plurality of circumferential rings 130 and loops formed at valleys of one or more circumferential rings of the plurality of circumferential rings 130. In some embodiments, circumferential rings that comprise loops formed at peaks and circumferential rings that comprise loops formed at valleys may alternate along the length of the endoprosthesis 100 and/or the body portion 110. In some embodiments, circumferential rings that comprise loops formed at peaks and circumferential rings that comprise loops formed at valleys may be mixed in various combinations that may repeat (e.g., form a pattern) or may not repeat (e.g., may be random) along the length of the endoprosthesis 100 and/or the body portion 110. Other configurations are also contemplated.

In some embodiments, the loop 140 formed at each peak (ref. 136) or at each valley (ref. 138) of each circumferential ring of the plurality of circumferential rings 130 is a closed loop that may be formed by crossing the single wire 120 over itself within each circumferential ring of the plurality of circumferential rings 130, as seen in Detail A. In some embodiments, a crossover point 122 of the single wire 120 forming the loop 140 and/or the closed loop may not be disposed at an apex 124 of its respective peak (ref. 136) or valley (ref. 138). In some embodiments, the (each) crossover point 122 of the single wire 120 forming the (each) loop 140 and/or the closed loop may be disposed opposite the apex 124 of its respective peak (ref. 136) or valley (ref. 138).

In at least some embodiments, a size (e.g., a perimeter and/or an internal area) of the loop 140 and/or the closed loop may be configured to increase when the endoprosthesis 100 and/or the body portion 110 is radially constrained (e.g., a collapsed delivery configuration), as shown in Detail A2, and the size (e.g., the perimeter and/or the internal area) of the loop 140 and/or the closed loop may be configured to decrease when the endoprosthesis 100 and/or the body portion 110 radially expands (e.g., an expanded deployed configuration), as shown in Detail A1. In some embodiments, the single wire 120 may be configured to translate and/or slide over and/or along itself such that the (each) crossover point 122 moves axially (e.g., toward the first end 112 and/or the second end 114) as the endoprosthesis 100 and/or the body portion 110 shifts between the collapsed delivery configuration and the expanded deployed configuration.

In some embodiments, various characteristics of the endoprosthesis 100 may be as discussed above and/or herein, such as, but not limited to, alignment of the peaks 136 and the valleys 138, the axial spacing 131 between adjacent pairs of circumferential rings of the plurality of circumferential rings 130, and/or the circumferential ring height.

In some embodiments, each circumferential ring of the plurality of circumferential rings 130 may be connected to an axially adjacent circumferential ring of the plurality of circumferential rings 130 by a transition segment 150 of the single wire 120, as seen in FIGS. 5-6 for example. Characteristics related to the transition segment(s) 150 may be the same as and/or substantially similar to those described above and/or herein.

In some embodiments, all transition segments (e.g., an array of transition segments consisting of each transition segment 150 connecting and/or extending between adjacent circumferential rings of the plurality of circumferential rings 130) of the single wire 120 may collectively define a transition zone 152. In some embodiments, the transition zone may extend parallel to the central longitudinal axis 102, as seen in FIG. 5. In some embodiments, each transition segment 150 of the single wire 120 may be aligned axially along the length of the body portion 110 and/or the endoprosthesis 100. In some embodiments, the transition zone may extend helically along and/or around the central longitudinal axis 102, as seen in FIG. 6.

In some embodiments, the endoprosthesis 100 may comprise a polymeric covering 160, shown in the figures via dotted shading, coupled to the body portion 110, the single wire 120, and/or the plurality of circumferential rings 130. In some embodiments, the polymeric covering 160 may be fixedly attached to the body portion 110, the single wire 120, and/or the plurality of circumferential rings 130.

In some embodiments, the polymeric covering 160 may extend along an inner surface of the endoprosthesis 100, the body portion 110, the single wire 120, and/or the plurality of circumferential rings 130. In some embodiments, the polymeric covering 160 may extend along an outer surface of the endoprosthesis 100, the body portion 110, the single wire 120, and/or the plurality of circumferential rings 130. In at least some embodiments, the body portion 110, the single wire 120, and/or the plurality of circumferential rings 130 may be embedded within the polymeric covering 160. Other configurations, including combinations thereof, are also contemplated. Some suitable but non-limiting examples of polymeric materials for the polymeric covering 160 are discussed below.

While not expressly shown, in some embodiments, an endoprosthesis system may comprise the endoprosthesis 100 and a delivery device. In some embodiments, the delivery device may comprise an inner tubular member and an outer tubular member. The endoprosthesis 100 may be disposable within an annular space disposed between the inner tubular member and the outer tubular member (e.g., within a lumen of the outer tubular member and around and/or radially outward of the inner tubular member) in a collapsed delivery configuration. The inner tubular member and the outer tubular member may be axially movable relative to each other to load and/or deploy the endoprosthesis 100. In some embodiments, the inner tubular member may comprise a guidewire lumen extending therein. In some embodiments, the delivery device may comprise a distal tip disposed at a distalmost end of the inner tubular member. In at least some embodiments, the guidewire lumen may extend through the distal tip. In alternative some embodiments, the delivery device may comprise a crochet delivery device. Other configurations are also contemplated.

In some embodiments, the endoprosthesis 100 may be configured to shift between the collapsed delivery configuration and an expanded deployed configuration. In some embodiments, the endoprosthesis 100 may be configured to self-expand from the collapsed delivery configuration to the expanded deployed configuration. In some embodiments, the endoprosthesis 100 may be balloon expandable from the collapsed delivery configuration to the expanded deployed configuration. Other configurations are also contemplated.

In some embodiments, the body portion 110 and/or the endoprosthesis 100 may be configured to minimize and/or avoid foreshortening and/or a change in the length of the body portion 110 and/or the endoprosthesis 100 as the body portion 110 and/or the endoprosthesis 100 shifts between the collapsed delivery configuration and the expanded deployed configuration. In some embodiments, the length of the body portion 110 and/or the endoprosthesis 100 from the first end 112 to the second end 114 may be configured to change by less than 10% when shifting between the collapsed delivery configuration and the expanded deployed configuration. In some embodiments, the length of the body portion 110 and/or the endoprosthesis 100 from the first end 112 to the second end 114 may be configured to change by less than 7.5% when shifting between the collapsed delivery configuration and the expanded deployed configuration. In some embodiments, the length of the body portion 110 and/or the endoprosthesis 100 from the first end 112 to the second end 114 may be configured to change by less than 5% when shifting between the collapsed delivery configuration and the expanded deployed configuration. In some embodiments, the length of the body portion 110 and/or the endoprosthesis 100 from the first end 112 to the second end 114 may be configured to change by less than 2.5% when shifting between the collapsed delivery configuration and the expanded deployed configuration. In some embodiments, the length of the body portion 110 and/or the endoprosthesis 100 from the first end 112 to the second end 114 may be configured to change by less than 1% when shifting between the collapsed delivery configuration and the expanded deployed configuration. Other configurations are also contemplated.

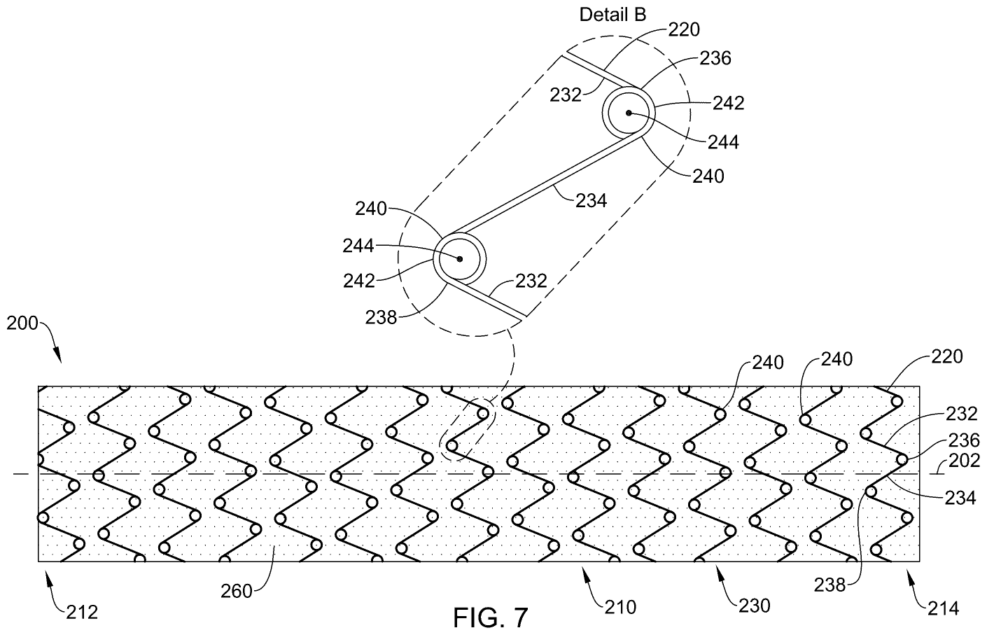

FIGS. 7-9 illustrate selected aspects of another example configuration of an endoprosthesis 200, such as a stent. The endoprosthesis 200 may comprise a body portion 210 extending axially from a first end 212 to a second end 214 along a central longitudinal axis 202. In some embodiments, the first end 212 and the second end 214 may define a length of the endoprosthesis 200 and/or the body portion 210. In at least some embodiments, the body portion 210 may be tubular. The body portion 210 may define a lumen extending therethrough from the first end 212 to the second end 214.

In some embodiments, the endoprosthesis 200 may comprise a flared first end portion (not shown) and/or a flared second end portion (not shown), wherein an outer diameter of the flared first end portion is greater than an outer diameter of the body portion 210 and/or an outer diameter of the flared second end portion is greater than the outer diameter of the body portion 210. Other configurations are also contemplated. The flared first end portion may be disposed proximate and/or adjacent the first end 212. In some embodiments, the flared first end portion may extend from the first end 212 to the body portion 210. The flared second end portion may be disposed proximate and/or adjacent the second end 214. In some embodiments, the flared second end portion may extend from the second end 214 to the body portion 210.

In some embodiments, the outer diameter of the body portion 210 may be generally uniform and/or generally constant along the length of the endoprosthesis 200 and/or the body portion 210. In some embodiments, the outer diameter of the body portion 210 may be generally uniform and/or generally constant along the length of the body portion 210 except for the flared first end portion and/or the flared second end portion, where present. In some embodiments, the outer diameter of the body portion 210 may be generally uniform and/or generally constant along the length of the body portion 210 from the flared first end portion to the flared second end portion, where present. In some embodiments, the outer diameter of the body portion 210 may be between about 4 millimeters and about 40 millimeters, between about 6 millimeters and about 35 millimeters, between about 8 millimeters and about 30 millimeters, between about 10 millimeters and about 25 millimeters, etc. Other configurations are also contemplated.

In some embodiments, the endoprosthesis 200 and/or the body portion 210 may be formed from a single wire 220 extending from the first end 212 to the second end 214. In some embodiments, the flared first end portion and/or the flared second end portion, where present, may be formed from the single wire 220. The single wire 220 may extend circumferentially around the central longitudinal axis 202 and/or the single wire 220 may encircle the central longitudinal axis 202. The single wire 220 is and/or consists of only one wire.

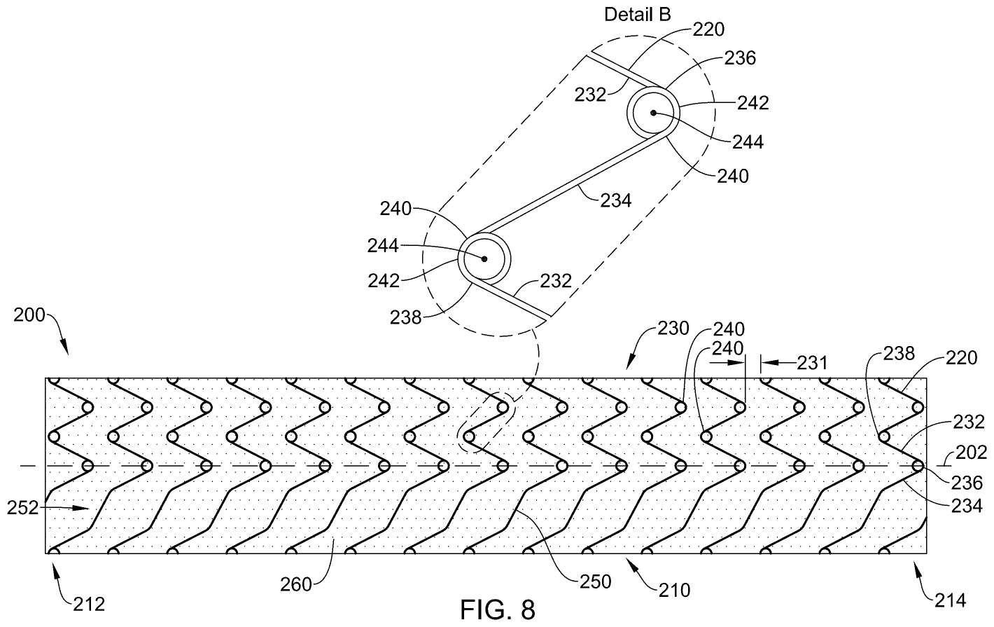

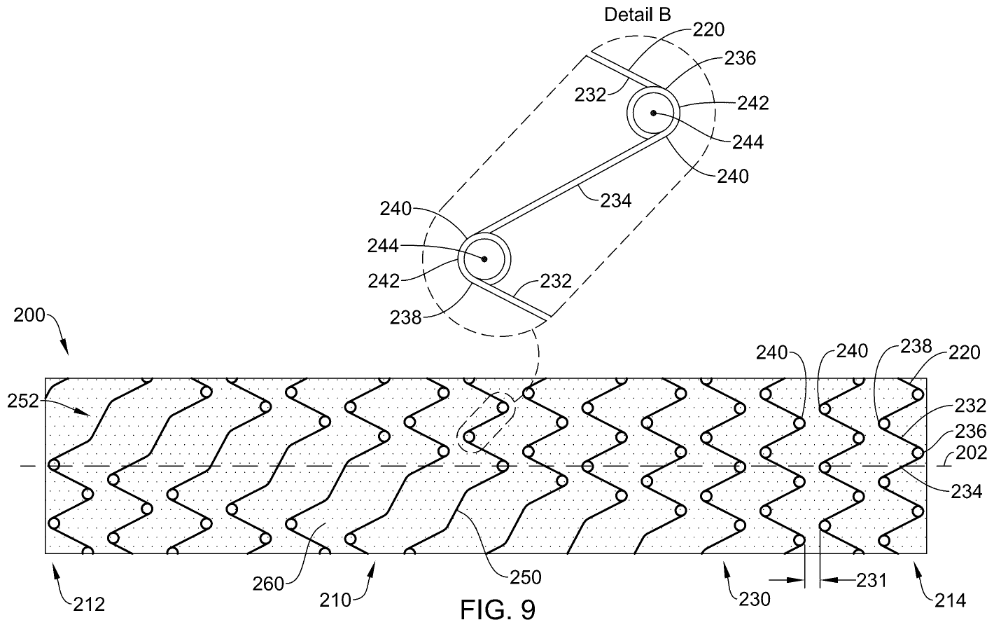

In some embodiments, the single wire 220 may form a plurality of circumferential rings 230 extending around the central longitudinal axis 202. In some embodiments, the plurality of circumferential rings 230 may extend helically around the central longitudinal axis 202, as seen in FIG. 7. In some embodiments, the plurality of circumferential rings 230 may extend non-helically around the central longitudinal axis 202, as seen in FIGS. 8-9. In some embodiments, the plurality of circumferential rings 230 may be substantially parallel to each other. Some suitable but non-limiting examples of materials for the endoprosthesis 200 and/or the single wire 220, such as metallic materials, composite materials, shape memory materials, combinations thereof, etc., are discussed below. In one non-limiting example, the endoprosthesis 200 and/or the single wire 220 may be formed from nickel-titanium alloy (e.g., nitinol). In another non-limiting example, the endoprosthesis 200 and/or the single wire 220 may be formed from nickel-titanium alloy with a platinum core. Other configurations and/or materials are also contemplated.

In some embodiments, the plurality of circumferential rings 230 may comprise a first end circumferential ring forming the first end 212, a second end circumferential ring forming the second end 214, and at least one medial circumferential ring disposed axially between the first end circumferential ring and the second end circumferential ring. In some embodiments, the at least one medial circumferential ring may comprise one circumferential ring, two circumferential rings, three circumferential rings, etc. up to a desired number of circumferential rings producing a desired overall length for the endoprosthesis 200.

In some embodiments, each circumferential ring of the plurality of circumferential rings 230 may comprise an undulating arrangement (e.g., a sinusoidal arrangement) of first struts 232 and second struts 234 defining peaks 236 and valleys 238. In at least some embodiments, within any particular circumferential ring of the plurality of circumferential rings 230, the first struts 232 have a first length defined by the peaks 236 and the valleys 238 and the second struts 234 have a second length defined by the peaks 236 and the valleys 238, wherein the first length is equal to the second length. In some embodiments, the first length and the second length may be uniform and/or constant among the plurality of circumferential rings 230. In some alternative embodiments, the first length and the second length may vary among the plurality of circumferential rings 230. In some embodiments, the first length and/or the second length may be between about 1 millimeter and about 15 millimeters. In some embodiments, the first length and/or the second length may be between about 3 millimeters and about 12 millimeters. In some embodiments, the first length and/or the second length may be between about 5 millimeters and about 10 millimeters. Other configurations are also contemplated.

In some embodiments, a loop 240 may be formed at each peak (ref. 236) and at each valley (ref. 238) of and/or within each circumferential ring of the plurality of circumferential rings 230. In some embodiments, some peaks 236 and/or some valleys 238 may be devoid of the loop 240. In some embodiments, peaks 236 and/or valleys 238 devoid of the loop 240 may form a pattern along the length of the endoprosthesis 200 and/or the body portion 210. In some embodiments, peaks 236 and/or valleys 238 devoid of the loop 240 may be random along the length of the endoprosthesis 200 and/or the body portion 210. Other configurations are also contemplated.

In some embodiments, the loop 240 formed at each peak (ref. 236) or at each valley (ref. 238) of each circumferential ring of the plurality of circumferential rings 230 is a closed loop that may be formed by crossing the single wire 220 over itself within each circumferential ring of the plurality of circumferential rings 230, as seen in Detail B. In some embodiments, a crossover point of the single wire 220 forming the loop 240 may be disposed at an apex 242 of its respective peak (ref. 236) or valley (ref. 238).

In some embodiments, the loop 240 and/or the closed loop formed at each peak (ref. 236) or at each valley (ref. 238) of each circumferential ring of the plurality of circumferential rings 230 may be formed by the single wire 220 extending at least 360 degrees around a center 244 of the loop 240. In some embodiments, the loop 240 and/or the closed loop formed at each peak (ref. 236) or at each valley (ref. 238) of each circumferential ring of the plurality of circumferential rings 230 may be formed by the single wire 220 extending at least 450 degrees around the center 244 of the loop 240.

In some embodiments, the single wire 220 extends in a first circumferential direction around the center 244 of the loop 240 at each peak (ref. 236) and in a second circumferential direction opposite the first circumferential direction around the center 244 of the loop 240 at each valley (ref. 238). In some embodiments, the single wire 220 extends in a counterclockwise direction around the center 244 of the loop 240 at each peak (ref. 236) and in a clockwise direction around the center 244 of the loop 240 at each valley (ref. 238). In some embodiments, the single wire 220 extends in a clockwise direction around the center 244 of the loop 240 at each peak (ref. 236) and in a counterclockwise direction around the center 244 of the loop 240 at each valley (ref. 238).

In some embodiments, an angle between and/or defined by the first struts 232 and the second struts 234 may be configured to close (e.g., reduce; become more acute) when the endoprosthesis 200 and/or the body portion 210 is radially constrained (e.g., a collapsed delivery configuration), and the angle between and/or defined the first struts 232 and the second struts 234 may be configured to open (e.g., increase; become less acute) when the endoprosthesis 200 and/or the body portion 210 radially expands (e.g., an expanded deployed configuration). In some alternative embodiments, a size (e.g., a perimeter and/or an internal area) of the loop 240 and/or the closed loop may be configured to increase when the endoprosthesis 200 and/or the body portion 210 is radially constrained (e.g., a collapsed delivery configuration), and the size (e.g., the perimeter and/or the internal area) of the loop 240 and/or the closed loop may be configured to decrease when the endoprosthesis 200 and/or the body portion 210 radially expands (e.g., an expanded deployed configuration).

In some embodiments, the peaks 236 and the valleys 238 of adjacent circumferential rings of the plurality of circumferential rings 230 may be aligned with each other axially along the length of the body portion 210 and/or the endoprosthesis 200, as seen in FIG. 8. For example, the peaks 236 of one circumferential ring of the plurality of circumferential rings 230 may be aligned axially with the peaks 236 of an immediately adjacent circumferential ring of the plurality of circumferential rings 230. Similarly, the valleys 238 of one circumferential ring of the plurality of circumferential rings 230 may be aligned axially with the valleys 238 of an immediately adjacent circumferential ring of the plurality of circumferential rings 230. In some embodiments, the peaks 236 and the valleys 238 of adjacent circumferential rings of the plurality of circumferential rings 230 may be aligned parallel to the central longitudinal axis 202. Other configurations are also contemplated.

In some embodiments, the peaks 236 and the valleys 238 of each circumferential ring of the plurality of circumferential rings 230 may be aligned with each other axially along the length of the body portion 210 and/or the endoprosthesis 200. For example, the peaks 236 of each circumferential ring of the plurality of circumferential rings 230 may be aligned axially with the peaks 236 of each other circumferential ring of the plurality of circumferential rings 230. Similarly, the valleys 238 of each circumferential ring of the plurality of circumferential rings 230 may be aligned axially with the valleys 238 of each other circumferential ring of the plurality of circumferential rings 230. In some embodiments, the peaks 236 and the valleys 238 of each circumferential ring of the plurality of circumferential rings 230 may be aligned parallel to the central longitudinal axis 202. Other configurations are also contemplated.

In some embodiments, loops (ref. 240) formed at each peak (ref. 236) of each circumferential ring of the plurality of circumferential rings 230 may be axially aligned with loops (ref. 240) formed at each peak (ref. 236) of each immediately adjacent circumferential ring of the plurality of circumferential rings 230 parallel to the central longitudinal axis 202, and loops (ref. 240) formed at each valley (ref. 238) of each circumferential ring of the plurality of circumferential rings 230 may be axially aligned with loops (ref. 240) formed at each valley (ref. 238) of each immediately adjacent circumferential ring of the plurality of circumferential rings 230 parallel to the central longitudinal axis 202. Other configurations are also contemplated.

In some embodiments, each adjacent pair of circumferential rings of the plurality of circumferential rings 230 may define an axial spacing 231 therebetween, as seen in FIGS. 8-9. The axial spacing 231 may be measured axially and/or parallel to the central longitudinal axis 202. In some embodiments, the axial spacing 231 may be defined between the valleys 238 of one circumferential ring and the peaks 236 of an immediately adjacent circumferential ring. In some embodiments, the axial spacing 231 between adjacent pairs of circumferential rings of the plurality of circumferential rings 230 may be uniform along the length of the body portion 210 and/or the endoprosthesis 200. In some embodiments, adjacent pairs of circumferential rings of the plurality of circumferential rings 230 are equally and/or uniformly spaced apart along the length of the body portion 210 and/or the endoprosthesis 200. In some embodiments, the axial spacing 231 between adjacent pairs of circumferential rings of the plurality of circumferential rings 230 may vary along the length of the body portion 210 and/or the endoprosthesis 200. In some embodiments, adjacent pairs of circumferential rings of the plurality of circumferential rings 230 are unequally and/or non-uniformly spaced apart along the length of the body portion 210 and/or the endoprosthesis 200. In some embodiments, the axial spacing 231 between adjacent pairs of circumferential rings of the plurality of circumferential rings 230 may increase along the length of the body portion 210 and/or the endoprosthesis 200 from the first end 212 toward and/or to the second end 214. Alternatively, in some embodiments, the axial spacing 231 between adjacent pairs of circumferential rings of the plurality of circumferential rings 230 may decrease along the length of the body portion 210 and/or the endoprosthesis 200 from the first end 212 toward and/or to the second end 214.

In some embodiments, the peaks 236 and the valleys 238 of each circumferential ring of the plurality of circumferential rings 230 may define a circumferential ring height for that circumferential ring measured parallel to the central longitudinal axis 202. In some embodiments, the circumferential ring height may be uniform along the length of the body portion 210 and/or the endoprosthesis 200 and/or among the plurality of circumferential rings 230, as illustrated in the figures. While not expressly shown, in some embodiments, the circumferential ring height may vary along the length of the body portion 210 and/or the endoprosthesis 200 and/or among the plurality of circumferential rings 230. In some embodiments, the circumferential ring height may increase along the length of the body portion 210 and/or the endoprosthesis 200 and/or among the plurality of circumferential rings 230 from the first end 212 toward and/or to the second end 214. Alternatively, in some embodiments, the circumferential ring height may decrease along the length of the body portion 210 and/or the endoprosthesis 200 and/or among the plurality of circumferential rings 230 from the first end 212 toward and/or to the second end 214.

In some embodiments, each circumferential ring of the plurality of circumferential rings 230 may be connected to an axially adjacent circumferential ring of the plurality of circumferential rings 230 by a transition segment 250 of the single wire 220, as seen in FIGS. 8-9 for example. In at least some embodiments, each circumferential ring of the plurality of circumferential rings 230 may be discontinuous and/or may extend around less than a full circumference of the endoprosthesis 200 and/or the body portion 210. Instead, each circumferential ring of the plurality of circumferential rings 230 may have a first end and a second end, wherein the transition segment 250 extends between a first end of one circumferential ring of the plurality of circumferential rings 230 and a second end of an immediately adjacent circumferential ring of the plurality of circumferential rings 230 (or the transition segment 250 extends between a second end of one circumferential ring of the plurality of circumferential rings 230 and a first end of an immediately adjacent circumferential ring of the plurality of circumferential rings 230). In some embodiments, at least a portion of the first end circumferential ring and/or the second end circumferential ring may extend completely around the full circumference of the endoprosthesis 200 and/or the body portion 210, and the at least one medial circumferential ring may be discontinuous and/or may extend around less than the full circumference of the endoprosthesis 200 and/or the body portion 210.

In some embodiments, the first struts 232 may be disposed at a first angle relative to the central longitudinal axis 202 in a side view and/or a flat pattern view of the body portion 210 and/or the endoprosthesis 200, and the second struts 234 may be disposed at a second angle relative to the central longitudinal axis 202 in the side view and/or the flat pattern view of the body portion 210 and/or the endoprosthesis 200. In at least some embodiments, the second angle may be different from the first angle. In some embodiments, the first angle and/or the second angle may be between zero degrees and 90 degrees. In some embodiments, the first angle and/or the second angle may be between 5 degrees and 60 degrees. In some embodiments, the first angle and/or the second angle may be between 10 degrees and 45 degrees. In some embodiments, the first angle and/or the second angle may be between 15 degrees and 30 degrees. Other configurations are also contemplated.

The transition segment 250 of the single wire 220 may be disposed at a third angle relative to the central longitudinal axis 202 in the side view and/or the flat pattern view of the body portion 210 and/or the endoprosthesis 200. In at least some embodiments, the third angle may be different from the first angle and the second angle. In some embodiments, the transition segment 150 may have a length between about 5 millimeters and about 20 millimeters. In some embodiments, the transition segment 150 may have a length between about 8 millimeters and about 17 millimeters. In some embodiments, the transition segment 150 may have a length between about 10 millimeters and about 15 millimeters. Other configurations are also contemplated. In some embodiments, the third angle may be between about 30 degrees and about 90 degrees. In some embodiments, the third angle may be between about 35 degrees and about 75 degrees. In some embodiments, the third angle may be between about 40 degrees and about 60 degrees. Other configurations are also contemplated.

In some embodiments, each circumferential ring of the plurality of circumferential rings 230 may be connected to an axially adjacent circumferential ring of the plurality of circumferential rings 230 by a transition segment 250 of the single wire 220 disposed nonparallel to the central longitudinal axis 202, the first struts 232, and the second struts 234 in the side view and/or the flat pattern view of the body portion 210 and/or the endoprosthesis 200. Other configurations are also contemplated.

In some embodiments, the transition segment 250 extending and/or disposed between axially adjacent circumferential rings of the plurality of circumferential rings 230 may extend between a valley (ref. 238) of one circumferential ring and a peak (ref. 236) of an immediately axially adjacent circumferential ring (or between a peak of one circumferential ring and a valley of an immediately axially adjacent circumferential ring). As illustrated, the transition segment(s) 250 extend from a peak (ref. 236) of a first circumferential ring disposed closer to the first end 212 to a valley (ref. 238) of a second circumferential ring immediately axially adjacent the first circumferential ring that is disposed closer to the second end 214 than the first circumferential ring. Other configurations are also contemplated. In some embodiments, the transition segment 250 may be disposed nonparallel with the first struts 232 and the second struts 234 of one circumferential ring (e.g., the first circumferential ring) and the first struts 232 and the second struts 234 of the immediately axially adjacent circumferential ring (e.g., the second circumferential ring). In some embodiments, the transition segment 250 may be a substantially straight or linear segment extending between a valley (ref. 238) of one circumferential ring and a peak (ref. 236) of an immediately axially adjacent circumferential ring (or between a peak of one circumferential ring and a valley of an immediately axially adjacent circumferential ring). In some embodiments, the transition segment 250 may be a non-linear segment extending between a valley (ref. 238) of one circumferential ring and a peak (ref. 236) of an immediately axially adjacent circumferential ring (or between a peak of one circumferential ring and a valley of an immediately axially adjacent circumferential ring)—for example, the transition segment 250 may have a bend formed along its length between two short linear segments. Other configurations are also contemplated.

In some embodiments, all transition segments (e.g., an array of transition segments consisting of each transition segment 250 connecting and/or extending between adjacent circumferential rings of the plurality of circumferential rings 230) of the single wire 220 may collectively define a transition zone 252. In some embodiments, the transition zone 252 may extend and/or may be arranged parallel to the central longitudinal axis 102, as seen in FIG. 8. In some embodiments, each transition segment 250 of the single wire 220 may be aligned axially along the length of the body portion 210 and/or the endoprosthesis 200. In some embodiments, the transition zone 252 may extend and/or may be arranged helically along and/or around the central longitudinal axis 202, as seen in FIG. 9.

In some embodiments, the peaks 236 and the valleys 238 of adjacent circumferential rings of the plurality of circumferential rings 230 may extend and/or may be arranged helically along the length of the body portion 210 and/or the endoprosthesis 200. In some embodiments, the peaks 236 and the valleys 238 of adjacent circumferential rings of the plurality of circumferential rings 230 may extend and/or may be arranged helically along and/or around the central longitudinal axis 202. In some embodiments, the peaks 236 and the valleys 238 of each circumferential ring of the plurality of circumferential rings 230 may extend and/or may be arranged helically along the length of the body portion 210 and/or the endoprosthesis 200. In some embodiments, the peaks 236 and the valleys 238 of each circumferential ring of the plurality of circumferential rings 230 may extend and/or may be arranged helically along and/or around the central longitudinal axis 202. Other configurations are also contemplated.

In some embodiments, loops (ref. 240) formed at each peak (ref. 236) of each circumferential ring of the plurality of circumferential rings 230 may extend and/or may be arranged helically along and/or around the central longitudinal axis 202, and loops (ref. 240) formed at each valley (ref. 238) of each circumferential ring of the plurality of circumferential rings 230 may extend and/or may be arranged helically along and/or around the central longitudinal axis 202. In some embodiments, loops (ref. 240) formed at each peak (ref. 236) of each circumferential ring of the plurality of circumferential rings 230 may be helically aligned around the central longitudinal axis 202 with loops (ref. 240) formed at each peak (ref. 236) of each immediately adjacent circumferential ring of the plurality of circumferential rings 230, and loops (ref. 240) formed at each valley (ref. 238) of each circumferential ring of the plurality of circumferential rings 230 may be helically aligned around the central longitudinal axis 202 with loops (ref. 240) formed at each valley (ref. 238) of each immediately adjacent circumferential ring of the plurality of circumferential rings 230. Other configurations are also contemplated.

In some embodiments, the endoprosthesis 200 may comprise a polymeric covering 260, shown in the figures via dotted shading, coupled to the body portion 210, the single wire 220, and/or the plurality of circumferential rings 230. In some embodiments, the polymeric covering 260 may be fixedly attached to the body portion 210, the single wire 220, and/or the plurality of circumferential rings 230.

In some embodiments, the polymeric covering 260 may extend along an inner surface of the endoprosthesis 200, the body portion 210, the single wire 220, and/or the plurality of circumferential rings 230. In some embodiments, the polymeric covering 260 may extend along an outer surface of the endoprosthesis 200, the body portion 210, the single wire 220, and/or the plurality of circumferential rings 230. In at least some embodiments, the body portion 210, the single wire 220, and/or the plurality of circumferential rings 230 may be embedded within the polymeric covering 260. Other configurations, including combinations thereof, are also contemplated. Some suitable but non-limiting examples of polymeric materials for the polymeric covering 260 are discussed below.

While not expressly shown, in some embodiments, an endoprosthesis system may comprise the endoprosthesis 200 and a delivery device. In some embodiments, the delivery device may comprise an inner tubular member and an outer tubular member. The endoprosthesis 200 may be disposable within an annular space disposed between the inner tubular member and the outer tubular member (e.g., within a lumen of the outer tubular member and around and/or radially outward of the inner tubular member) in a collapsed delivery configuration. The inner tubular member and the outer tubular member may be axially movable relative to each other to load and/or deploy the endoprosthesis 200. In some embodiments, the inner tubular member may comprise a guidewire lumen extending therein. In some embodiments, the delivery device may comprise a distal tip disposed at a distalmost end of the inner tubular member. In at least some embodiments, the guidewire lumen may extend through the distal tip. In alternative some embodiments, the delivery device may comprise a crochet delivery device. Other configurations are also contemplated.

In some embodiments, the endoprosthesis 200 may be configured to shift between the collapsed delivery configuration and an expanded deployed configuration. In some embodiments, the endoprosthesis 200 may be configured to self-expand from the collapsed delivery configuration to the expanded deployed configuration. In some embodiments, the endoprosthesis 200 may be balloon expandable from the collapsed delivery configuration to the expanded deployed configuration. Other configurations are also contemplated.

In some embodiments, the body portion 210 and/or the endoprosthesis 200 may be configured to minimize and/or avoid foreshortening and/or a change in the length of the body portion 210 and/or the endoprosthesis 200 as the body portion 210 and/or the endoprosthesis 200 shifts between the collapsed delivery configuration and the expanded deployed configuration. In some embodiments, the length of the body portion 210 and/or the endoprosthesis 200 from the first end 212 to the second end 214 may be configured to change by less than 10% when shifting between the collapsed delivery configuration and the expanded deployed configuration. In some embodiments, the length of the body portion 210 and/or the endoprosthesis 200 from the first end 212 to the second end 214 may be configured to change by less than 7.5% when shifting between the collapsed delivery configuration and the expanded deployed configuration. In some embodiments, the length of the body portion 210 and/or the endoprosthesis 200 from the first end 212 to the second end 214 may be configured to change by less than 5% when shifting between the collapsed delivery configuration and the expanded deployed configuration. In some embodiments, the length of the body portion 210 and/or the endoprosthesis 200 from the first end 212 to the second end 214 may be configured to change by less than 2.5% when shifting between the collapsed delivery configuration and the expanded deployed configuration. In some embodiments, the length of the body portion 210 and/or the endoprosthesis 200 from the first end 212 to the second end 214 may be configured to change by less than 1% when shifting between the collapsed delivery configuration and the expanded deployed configuration. Other configurations are also contemplated.

The materials that can be used for the various components of the endoprosthesis and/or the endoprosthesis system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the delivery device, the endoprosthesis, the single wire, the polymeric covering, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-clastic and/or super-clastic nitinol; other nickel alloys such as nickel-chromium-molybdenum alloys (e.g., UNS: N06625 such as INCONEL® 625, UNS: N06022 such as HASTELLOYR C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An endoprosthesis, comprising:
a body portion extending axially from a first end to a second end along a central longitudinal axis;
wherein the body portion is formed from a single wire extending from the first end to the second end;
wherein the single wire forms a plurality of circumferential rings extending around the central longitudinal axis;
wherein each circumferential ring of the plurality of circumferential rings comprises an undulating arrangement of struts defining peaks and valleys;
wherein at least one circumferential ring of the plurality of circumferential rings comprises a loop formed at each peak or at each valley of the at least one circumferential ring of the plurality of circumferential rings.

2. The endoprosthesis of claim 1, wherein the plurality of circumferential rings extends helically around the central longitudinal axis.

3. The endoprosthesis of claim 1, wherein the loop formed at each peak or at each valley is a closed loop formed by crossing the single wire over itself within the at least one circumferential ring of the plurality of circumferential rings.

4. The endoprosthesis of claim 1, further comprising a polymeric covering coupled to the body portion.

5. The endoprosthesis of claim 1, wherein a crossover point of the single wire forming the loop is disposed opposite an apex of its respective peak or valley.

6. The endoprosthesis of claim 1, wherein the plurality of circumferential rings extends non-helically around the central longitudinal axis.

7. An endoprosthesis, comprising:
a body portion extending axially from a first end to a second end along a central longitudinal axis;
wherein the body portion is formed from a single wire extending from the first end to the second end;
wherein the single wire forms a plurality of circumferential rings extending around the central longitudinal axis;
wherein each circumferential ring of the plurality of circumferential rings comprises an undulating arrangement of struts defining peaks and valleys;
wherein a loop is formed at each peak or at each valley of each circumferential ring of the plurality of circumferential rings.

8. The endoprosthesis of claim 7, wherein the plurality of circumferential rings extends helically around the central longitudinal axis.

9. The endoprosthesis of claim 7, wherein the loop formed at each peak or at each valley is a closed loop formed by crossing the single wire over itself within each circumferential ring of the plurality of circumferential rings.

10. The endoprosthesis of claim 9, wherein a crossover point of the single wire forming the closed loop is not disposed at an apex of its respective peak or valley.

11. The endoprosthesis of claim 9, wherein a size of the closed loop is configured to increase when the endoprosthesis is radially constrained, and the size of the closed loop is configured to decrease when the endoprosthesis radially expands.

12. The endoprosthesis of claim 7, further comprising a polymeric covering coupled to the body portion.

13. An endoprosthesis, comprising:
a body portion extending axially from a first end to a second end along a central longitudinal axis;
wherein the body portion is formed from a single wire extending from the first end to the second end;
wherein the single wire forms a plurality of circumferential rings extending around the central longitudinal axis;
wherein each circumferential ring of the plurality of circumferential rings comprises an undulating arrangement of struts defining peaks and valleys;
wherein a loop is formed at each peak and at each valley of each circumferential ring of the plurality of circumferential rings.

14. The endoprosthesis of claim 13, wherein loops formed at each peak of each circumferential ring are axially aligned with loops formed at each peak of each immediately adjacent circumferential ring parallel to the central longitudinal axis, and loops formed at each valley of each circumferential ring are axially aligned with loops formed at each valley of each immediately adjacent circumferential ring parallel to the central longitudinal axis.

15. The endoprosthesis of claim 13, wherein loops formed at each peak of each circumferential ring are helically aligned around the central longitudinal axis with loops formed at each peak of each immediately adjacent circumferential ring, and loops formed at each valley of each circumferential ring are helically aligned around the central longitudinal axis with loops formed at each valley of each immediately adjacent circumferential ring.

16. The endoprosthesis of claim 13, wherein the loop formed at each peak and at each valley of each circumferential ring is formed by the single wire extending at least 360 degrees around a center of the loop.

17. The endoprosthesis of claim 16, wherein the loop formed at each peak and at each valley of each circumferential ring is formed by the single wire extending at least 450 degrees around the center of the loop.

18. The endoprosthesis of claim 16, wherein the single wire extends in a first circumferential direction around the center of the loop at each peak and in a second circumferential direction opposite the first circumferential direction around the center of the loop at each valley.

19. The endoprosthesis of claim 13, wherein a crossover point of the single wire forming the loop is disposed at an apex of its respective peak or valley.

20. The endoprosthesis of claim 13, further comprising a polymeric covering coupled to the body portion.
